(19) United States
(12) Patent Application Publication
(10) Pub. No.: US 2006/0004035 A1
(43) Pub. Date: Jan. 5, 2006

(54) System for identification of a pharmaceutical product

(75) Inventors: Charles M. Barr, Salt Lake City, UT (US); Brian Hague, Bountiful, UT (US)

Correspondence Address: CEPHALON, INC., 41 MOORES ROAD, PO BOX 4011, FRAZER, PA, 19355, US

(73) Assignee: Cephalon, Inc., Frazer, PA (US)

(21) Appl. No.: 11/166,638

(22) Filed: Jun. 24, 2005

Related U.S. Application Data
(60) Provisional application No. 60/582,901, filed on Jun. 25, 2004.

(51) Int. Cl. A61K 31/485; A61K 31/445

(52) U.S. Cl. 514/282; 514/317; 604/1

(57) ABSTRACT

The present invention relates to a system for identification for pharmaceutical products, and more particularly to a system for identification of an analgesic dosage unit. The invention relates directly to a system for providing direct safety at the point of use of a pharmaceutical product.

### FIELD OF THE INVENTION

The present invention relates to a system for identification for pharmaceutical products, and more particularly to a system for identification of an analgesic dosage unit. The invention relates directly to a system for providing direct safety at the point of use of pharmaceutical products.

### BACKGROUND OF THE INVENTION

Solid pharmaceutical dosage forms are well known in the art. Compared to other dosage forms, oral solid dosage forms are the most preferred dosage forms and account for greatest percent of all the pharmaceutical products on the market. Solid dosage forms are easier for a patient or caregiver to identify, handle and administer. They are also non-invasive and have high patient compliance.

Solid dosage forms can be further divided into several groups, based upon the route by which the drug is delivered, including, for example, gastrointestinal (GI) tract delivery, suppository delivery and oral transmucosal delivery. The majority of solid dosage forms on the market are designed for gastro-intestinal delivery. GI delivery is often referred to simply as “oral delivery,” because a tablet or capsule is initially introduced orally, and swallowed. However, once swallowed, identification of the drug being delivered from a safety perspective is a moot point. Relatively few drug formulations are designed as solid dosage forms intended to deliver a drug through the oral mucosa wherein identification of the drug being delivered from a safety perspective is still feasible.

Despite the overall popularity of other delivery methods, oral transmucosal (OT) delivery is a particularly advantageous delivery route. One of the advantages of OT delivery is that it is non-invasive. Furthermore, OT delivery generally has better patient compliance, less risk of infection and lower cost than invasive procedures such as injection and implantation. It also has much shorter onset time, i.e., the time from administration to therapeutic effect, than does oral delivery. Oral transmucosal delivery is simple and can be administered by the caregiver or the patient with minimal discomfort.

Various solid dosage forms have been used to deliver drugs via the oral mucosal tissue. A lozenge with an integrated oral transmucosal applicator, (hereinafter, a “lozenge-on-a-stick”) dosage form of transmucosal drug delivery is disclosed in U.S. Pat. No. 4,671,953 to Stanley, et al. In addition to being non-invasive and providing a particularly easy method of delivery, the lozenge-on-a-stick dosage form allows a patient or caregiver to move the dosage form in and out of the mouth to titrate the dose. This practice is called dose-to-effect, in which a patient or caregiver controls the administration of the dose until the expected therapeutic effect is achieved. This is particularly important for certain symptoms, such as pain, nausea, motion sickness, and premedication prior to anesthesia because each patient needs a different amount of medication to treat these symptoms. For these types of treatments, the patient is best suited to ascertain the appropriate dose of medication to be administered. Once the appropriate amount of drug is delivered, the patient or caregiver can remove the lozenge-on-a-stick, thus, stopping delivery of the drug. Conversely, the stick provides a simple mechanism for the patient to re-insert the dosage form to continue delivery of the drug. This feature is especially important for particularly potent drugs, which may present a significant advantage of terminating drug administration once the desired effect is achieved.

In addition to the disclosure of U.S. Pat. No. 4,671,953, a lozenge-on-a-stick dosage form of oral transmucosal drug delivery is also disclosed in U.S. Pat. No. 4,863,737 to Stanley, et al.; U.S. Pat. No. 5,132,114, to Stanley, et al.; U.S. Pat. No. 5,785,989, to Stanley, et al.; U.S. Pat. No. 5,288,498, to Stanley, et al.; U.S. Pat. No. 5,288,497, to Stanley, et al.; U.S. Pat. No. 5,855,908, to Stanley, et al.; and pending U.S. patent application Ser. No. 10/,771,046, to Hague, et al., filed Feb. 3, 2004. However, none of these prior art references disclose the system of the present invention, i.e., the use of multiple indicia for identification of an analgesic dosage unit.

A particularly successful example of the lozenge-on-a-stick-type oral transmucosal solid dosage form is the ACTIQ® brand of oral transmucosal fentanyl citrate, that has been marketed in the United States and abroad for several years. In one ACTIQ® brand product, the active ingredient, fentanyl citrate, is intermixed in a sugar-based excipient ENDEX® (spray-crystallized maltose-dextrose magnesium stearate porous spheres), and compressed to produce what is essentially a drug-containing lozenge, to which a stick has been affixed. The product is approved by the United States Food and Drug Administration (FDA) for the control of break-through pain in opioid tolerant cancer patients. ACTIQ® is available in several strengths, and patients may regulate the amount of drug that is administered by varying the extent to which the product is rubbed over the oral mucosal surfaces, and the duration of administration. Through repeated usage, patients develop an understanding of just how much of the product will be needed to manage their pain. Since fentanyl is a very powerful narcotic, it is imperative for the manufacturers of the oral transmucosal fentanyl citrate product to minimize the risks of administering the prescription product.

To minimize the risk of administering prescription drugs, pharmaceutical products are sold with extensive labeling. Pharmaceutical packaging is provided with instructions, warnings, and other information. Goods which can be packaged as unit doses of medicaments are packaged with written instructions inside the box or other package which contains the unit doses of the medicaments. These unit doses of the medicaments are intended for use by the patient at the bedside or other place of treatment and may be readily separated from the written instructions inside the box. Every effort is made to insure that the instructions are followed at the point of administration.

To minimize the risks associated with administering a lozenge-on-a-stick, for example ACTIQ®, labeling the lozenge-on-a-stick with safety indicia is imperative. In order to understand the background of the present invention and the environment in which it is intended to be used, it is helpful to visualize a patient self administering a lozenge-on-a-stick in order to induce a sedative, analgesic or anesthetic effect. Such a patient may become drowsyor incoherent during or after the administration process. A problem arises when the patient obtains or exceeds the desired dose to effect and the caregiver or a third party must be able to identify the pharmaceutical product administered post facto. A problem also arises if the lozenge-on-a-stick has not been fully consumed and the patient does not properly dispose of any unused portion of the medication.

Childproof containers for the interim storage of medicated oral transmucosal dosage forms are disclosed in U.S. Pat. Nos. 6,173,851, and 6,286,698, both to Hague, et al. In addition to childproof containers, these patents disclose a lozenge-on-a-stick wherein the stick includes a bisecting paddle with the warning “Rx” disclosed thereon. These patents do not disclose the handle of the present invention, nor do they disclose the multiple indicia of the present invention as further described herein.

If the lozenge-on-a-stick does not contain appropriate product identifying information post administration then an unnecessary risk is taken. Likewise, if all product identifying information can be separated from the lozenge-on-a-stick, thereby losing all identifying information, an unnecessary risk is taken. Without a system for product identification of an analgesic dosage unit at the point of use of pharmaceutical products, improper administration or disposal of analgesic products can cause serious harm to the patients being treated and others who may mistakenly consume the product.

Accordingly, it is an object of this invention to provide additional safety at the point of use of oral transmucosal products which induce a sedative, analgesic or anesthetic effect in the patient.

It is an object of this invention to provide a system for pharmaceutical product identification which can be used at the point of administration to ensure that proper identification of the product and other information be communicated to health care personnel should a situation arise which may cause serious harm to the patient being treated or others.

It is another object of this invention to provide a handle at the end of the stick opposite of the drug-containing matrix and to make the handle different set of dimensions than the stick. This allows for ease of handling, greater control and improved patient compliance of oral transmucosal delivery of the drug by the patient. It also differentiates the pharmaceutical dosage unit from a common lollipop, a safety feature designed to make the pharmaceutical dosage unit less attracting to and/or recognized by a child as a consumable lollipop. It may also provide for the pharmaceutical dosage unit to have a blunt end, a safety feature which prevents its use as a pointed device capable of opening the pharmaceutical packaging of a new pharmaceutical dosage unit.

### SUMMARY OF THE INVENTION

It has been discovered that the above and other objects of the present invention may be accomplished in the following manner.

Specifically, a new system for identification of a pharmaceutical dosage unit has been discovered where the drug is intended to induce a sedative, analgesic or anesthetic effect in a patient after oral transmucosal delivery of the drug.

The new system includes a pharmaceutical dosage unit comprising, a drug-containing matrix being secured or attached to the first end of a stick, wherein the drug-containing matrix displays a first identifying indicia on the surface of the drug-containing matrix, a handle included on the opposing second end of the stick, wherein the handle displays a second identifying indicia, and a label adhered to the surface of the handle, wherein the label displays a third identifying indicia. The first, second and third identifying indicia cooperatively convey information to the user of the pharmaceutical dosage unit. Preferably the new system includes a pharmaceutical dosage unit for use in the oral transmucosal of a drug to a patient, in order to induce a sedative, analgesic or anesthetic effect in the patient.

Examples of information which is cooperatively conveyed by the first, second, and third indicia are safety information, product identifying information, dosage information, restriction on use warnings, color codings and other information. In addition, the indicia may convey contents, generic name, brand name, sources of goods, dosage strengths and other information. In addition, the first, second, and third indicia may have identical messages so as to create a failsafe redundancy in the information conveyed.

### BRIEF DESCRIPTION OF THE DRAWINGS

These and other objects of the present invention and the various features and details of the operation and construction thereof are hereinafter more fully set forth with reference to the accompanying drawings, where:

FIG. 1 is a front elevational view of a pharmaceutical dosage unit shown without indicia.

FIG. 2 is a front elevational view of a pharmaceutical dosage unit shown with first identifying indicia and third identifying indicia.

FIG. 2A is a label from a pharmaceutical dosage unit shown with third identifying indicia.

FIG. 3A is a front elevational view of a pharmaceutical dosage unit shown with first identifying indicia and second identifying indicia.

FIG. 3B is a front elevational view of a pharmaceutical dosage unit shown with first identifying indicia and alternative second identifying indicia.

FIG. 4 is an exploded elevational view of a pharmaceutical dosage unit shown with first identifying indicia printed on a drug-containing matrix secured or attached to a stick including a handle identified with second indicia and an exploded view of the label including third identifying indicia.

FIG. 5A is a perspective view of a pharmaceutical dosage unit shown with a barrel handle.

FIG. 5B is a perspective view of a pharmaceutical dosage unit shown with a rectangular box handle.

### DETAILED DESCRIPTION OF THE PREFERRED EMBODIMENT

The present application is directed to compositions and methods for administering same by oral transmucosal delivery. “Oral transmucosal delivery” refers to the delivery of a pharmaceutical agent across a mucous membrane in the oral cavity, pharyngeal cavity, or esophagus, and may be contrasted, for example, with traditional oral delivery, in which absorption of the drug occurs in the stomach and/or intestines. Accordingly, routes of administration in which the pharmaceutical agent is absorbed through the buccal, sublingual, gingival, pharyngeal, and/or esophageal mucosa are all encompassed within “oral transmucosal delivery,” as that term is used herein. Preferably, oral transmucosal delivery involves the administration of an oral transmucosal solid dosage form to the oral cavity of a patient, which is held in the oral cavity and dissolved, thereby releasing the pharmaceutical agent or drug for oral transmucosal delivery. Of course, as the solid dosage form dissolves in the oral cavity, some of the saliva containing the pharmaceutical agent may be swallowed, and a portion of the drug may ultimately be absorbed from the stomach and/or intestines.

As used herein, the term “pharmaceutical dosage unit” broadly refers to a lozenge-on-a-stick, which comprises the drug-containing matrix (i.e. lozenge) affixed to a stick with a handle. The drug-containing matrix may be held between the cheek and gum or placed on or under the tongue, or it may be actively licked, sucked, or rubbed across the oral mucosa by the patient or a caregiver. Preferably, the solid dosage form is not bitten or chewed, unless the broken pieces are then held in the mouth until substantially dissolved.

As used herein, the term “drug-containing matrix” broadly refers to the lozenge component of the lozenge-on-a-stick. The lozenge component comprises a suitable drug dispersed within a soluble carbohydrate matrix, a soluble carbohydrate free matrix, or other suitable soluble pharmaceutical matrix capable of oral transmucosal delivery of the drug to the patient. Examples of lozenges are disclosed in U.S. Pat. Nos. 4,863,737; 5,132,114; 5,785,989; 5,288,498; 5,288,497; and 5,855,908; and pending U.S. patent application Ser. No. 10/,771,046, to Hague, et al., filed Feb. 3, 2004.

As used herein, the term “handle” broadly refers to that part of the pharmaceutical dosage unit designed to be held or operated with the hand or fingers. The handle is at one end of the stick opposite of the drug-containing matrix and conveys second indicia in a permament manner. The handle may be molded as part of the stick or may be attached to the stick. The handle is different in dimensions than the stick and has an identifiable shape distinct from the stick. Examples of a handle include, but are not limited to, cyclinder (or barrel), cube, rectangular box (tetragonal prism), triangular prism, pentagonal prism, and hexagonal prism. Preferred is cyclinder.

As described above, the handle conveys second indicia in a permanent manner. Example, known in the art, of a permanent manner are embossed indicia and debossed indicia. Second indicia, whether embossed or debossed, may be prepared either as the handle is manufactured or after the handle is manufactured. As used herein, the term “embossed” is intended to mean the indicia are recessed into the surface of the handle. As such, the term “embossed”, includes, but is not limited to, indicia which are engraved, etched, and/or burned into the surface of the handle. The term “embossed” also includes indicia which are molded into the handle at the time of manufactuirng the handle, for example, if the handle is manufactured by injection molding.

As used herein, the term “generic name” means the established name of the active drug ingredient as defined under section 505(e)(3) of the Federal Food, Drug, and Cosmetic Act. Most generic names originate from nonproprietary names for drug substances.

The term “patient,” as used herein, refers to animals, including mammals, preferably humans.

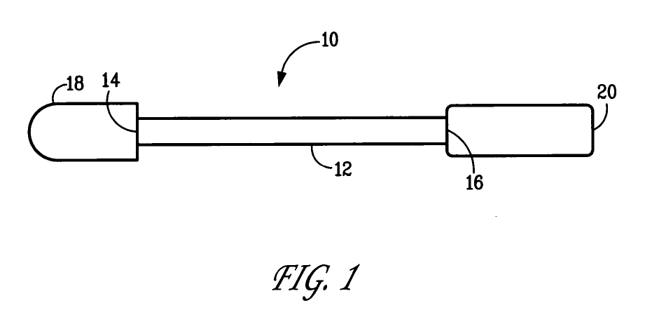

As shown in FIG. 1, the system of the present invention for pharmaceutical product identification of a pharmaceutical dosage unit includes a device, shown generally by the numeral 10. The pharmaceutical dosage unit 10 includes a stick 12 having a top end 14 and a bottom end 16. A consumable drug-containing matrix 18, on which a first identifying indicia (not shown) is placed, is secured or attached to the top end 14 of the stick 12. The pharmaceutical dosage unit 10 also includes a handle 20, on which a second identifying indicia (not shown) is placed, at the bottom end of the stick 12. Among other parts of the pharmaceutical dosage unit 10 is a label 22 adhered to the surface of the handle 20, on which a third identifying indicia (not shown) is placed.

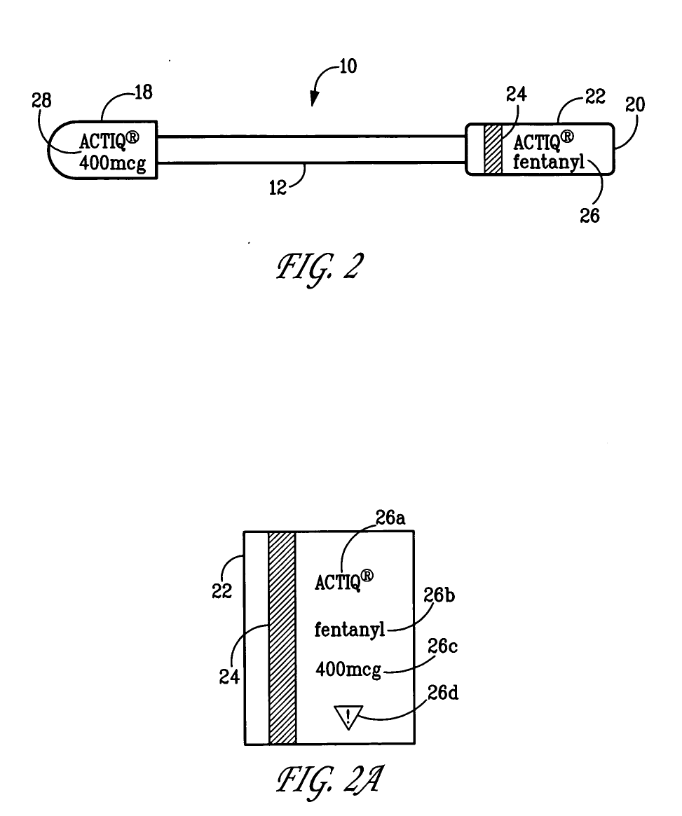

Shown in FIG. 2 is a pharmaceutical identification system, as might be obtained from the pharmaceutical packaging of the product, in which pharmaceutical dosage unit 10 has a label 22 adhered to the handle 20. On the label 22, is placed a third identifying indicia 24 and 26 wherein the third identifying indicia includes a color coding indicia 24 in addition to the brand name and generic name 26 of the pharmaceutical dosage unit. Color coding indicia 24 of the label 22 can reflect particular dosage strengths of the dosage units. On the drug-containing matrix 18, is placed a first identifying indicia 28 which, for illustrative purposes, may include the brand name and dosage strength of the pharmaceutical dosage unit. The information 28 contained on the pharmaceutical dosage unit 10, to be seen before consumption of the unit by the patient, should correlate with the information 26 such that the patient, caregiver, emergency attendant, or any other attendant using the system will have whatever information as need be after the pharmaceutical dosage unit 10 is either consumed or partially consumed to identify the pharmaceutical dosage unit used.

In FIG. 2, whether color coding or other information is important for the user, that information on the label 22 can be conveyed without the possibility of the information being dissolved by consumption of the product. Similarly, color coding indicia 24 may be matched or coordinated with a similar color coding provided on the packaging and/or containers filled with instructions, warnings, and other information from which the product comes originally packaged in.

Shown in FIG. 2A is an example of a label 22 from a pharmaceutical dosage unit wherein some but not all of the indicia correlates to first indicia and to second indicia. On the label 22, is placed third indicia including color coding 24, brand name 26a, generic name 26b, pharmaceutical dosage unit 26c, and a yellow triangle with an exclamation point printed inside of the triangle 26d.

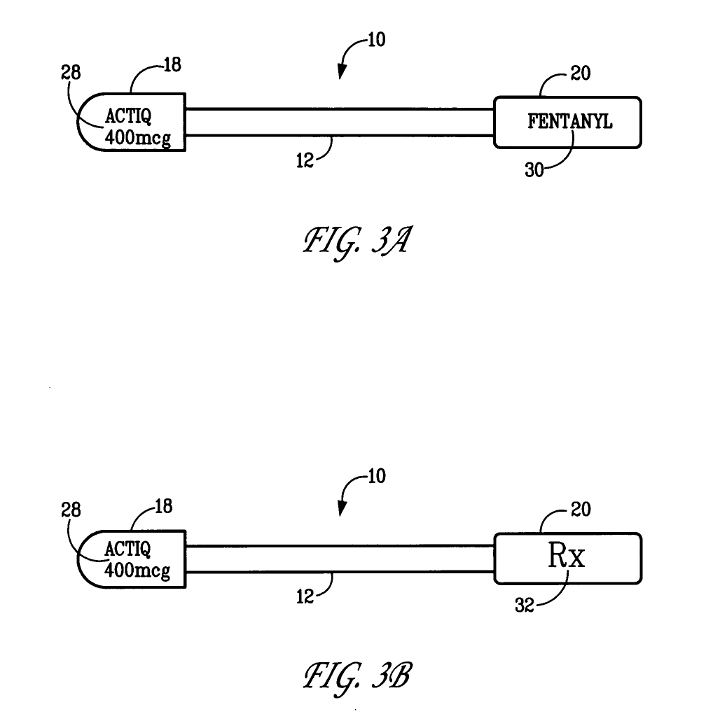

Shown in FIG. 3A is a pharmaceutical identification system in which the label 22 has been removed from the handle 20 of the pharmaceutical dosage unit 10. On the handle 20 is embossed second identifying indicia 30 wherein the second identifying indicia includes the generic name of the pharmaceutical dosage unit. On the drug-containing matrix 18, is placed a first identifying indicia 28 which, for illustrative purposes, may include the brand name and dosage strength of the pharmaceutical dosage unit. The information 30 contained on the pharmaceutical dosage unit 10, to be seen if the label 22 is removed from the dosage unit 10 before, during or after consumption of the unit by the patient, should correlate with the information 26, such that the patient, caregiver, emergency attendant, or any other attendant using the system will have whatever information as needed to identify the pharmaceutical dosage unit used after the pharmaceutical dosage unit 10 is either consumed or partially consumed.

Shown in FIG. 3B is another example of a pharmaceutical identification system in which the label 22 has been removed from the handle 20 of the pharmaceutical dosage unit 10. On handle 20 is embossed second identifying indicia 32 wherein the second identifying indicia includes restriction on use warnings of the pharmaceutical dosage unit. On the drug-containing matrix 18, is placed a first identifying indicia 28 which, for illustrative purposes, may include the brand name and dosage strength of the pharmaceutical dosage unit. The information 32 contained on the pharmaceutical dosage unit 10, to be seen if the label 22 is removed from the dosage unit 10 before, during or after consumption of the unit by the patient, should correlate with the information 26 such that the patient, caregiver, emergency attendant, or any other attendant using the system will have whatever information as needed to identify the pharmaceutical dosage unit used after the pharmaceutical dosage unit 10 is either consumed or partially consumed.

Alternatively, the second indicia embossed on handle 20 may have both the generic name formulated into the pharmaceutical dosage unit as well as restriction on use warnings. For example, the handle may have embossed on it “fentanyl” and “Rx”.

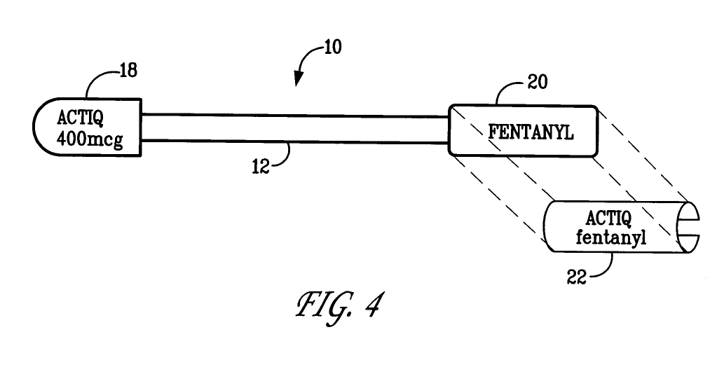

Shown in FIG. 4 is a pharmaceutical identification system in which pharmaceutical dosage unit 10 includes a stick 12, consumable drug-containing matrix 18, on which first identifying indicia is placed, a handle 20, on which a second identifying indicia is placed, and a label 22, on which a third identifying indicia is placed. In FIG. 4 the label 22 is shown in an exploded view for illustrative purposes, however, in the pharmaceutical identification system of the invention the label 22 is adhered to the surface of the handle 20.

Shown in FIG. 2 and in FIG. 4 is a pharmaceutical identification system, as might be obtained from the pharmaceutical packaging of the product, in which pharmaceutical dosage unit 10 has a label 22 adhered to the handle 20. In FIG. 2 and in FIG. 4 when the label 22 adhers to the handle 20, it is intended to cover the second indicia of the handle 20. However, in an alternative embodiment the second indicia may appear on the handle in a postion not covered by the label, for example when the handle is of sufficient dimension to place the second indicia and third indicia in non overlapping locations. Alternatively, the second indicia may occur on the stick as long as it does not alter the general structure and/or dimensions of the stick. A preferred stick is a cylindrical rod.

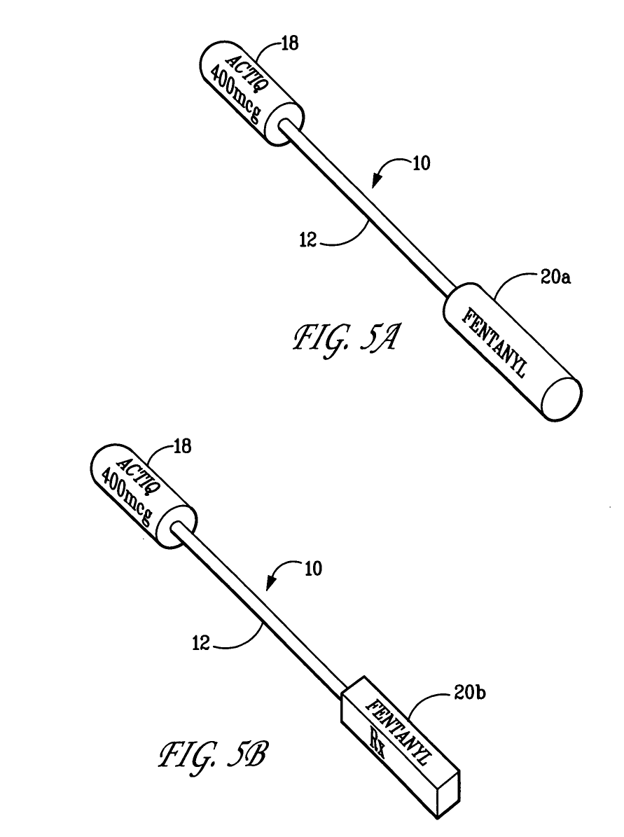

Shown in FIG. 5A is a pharmaceutical identification system in which pharmaceutical dosage unit 10 includes a stick 12, consumable drug-containing matrix 18, on which first identifying indicia is placed, and a barrel handle 20a, on which second identifying indicia is placed. For purposes of illustration the label 22, on which third identifying indicia is placed, is not shown.

Shown in FIG. 5B is a pharmaceutical identification system in which pharmaceutical dosage unit 10 includes a stick 12, consumable drug-containing matrix 18, on which first identifying indicia is placed, and a rectangular box handle 20b, on which second identifying indicia is placed. For purposes of illustration the label 22, on which third identifying indicia is placed, is not shown.

In each case, the information conveyed by the first identifying indicia on the drug-containing matrix is cooperatively combined with the information conveyed by the second identifying indicia on handle and the third identifying indicia on the label, to provide a failsafe redundancy of information at the point of use of the pharmaceutical dosage unit to which it is applied.

As has been noted, drug product information, brand name information, generic name information, dosage strength information, restrictions on use information, such as the appropriate information for unambiguous identification of a prescription drug substance in the product can be conveyed with both the first, second, and third indicia, either duplicating the information or combining to convey that information. Extra assurances are given when the same message is on both the drug-containing matrix and the label, for example “ACTIQ” and dosage strength. Extra assurances are given when the same message is on both the handle and the label, for example “fentanyl”. Similarly, restrictions on use or other warnings can be used, for example “Rx” or a yellow triangle with an exclamation point drawn within the triangle.

Preferred embodiments of first indicia of the present invention include the brand name of the drug and dosage strength information. A preferred example of the brand name is “ACTIQ®”. Preferred examples of the dosage strengths are “200 mcg”, “400 mcg”, “600 mcg”, “800 mcg”, “1200 mcg”, and “1600 mcg”.

Preferred embodiments of second indicia of the present invention include the generic name of the drug and restrictions on use or other warning information. A preferred example of the generic name is “fentanyl”. A preferred example of a restriction on use or other warning information is “Rx”.

Preferred embodiments of third indicia of the present invention include brand name information, generic name information, dosage strength information, warning information, and color coding. A preferred example of third indicia is “ACTIQ®”; “fentanyl”; a yellow triangle with an exclamation point printed in it; a dosage strength selected from “200 mcg”, “400 mcg”, “600 mcg”, “800 mcg”, “1200 mcg”, and “1600 mcg”; and color coding correlating to the dosage strength that is printed on the label.

It is particularly important that the product be immediately and visually identified. Messages, instructions or warnings must be highly visible and for that reason the printing process must be sufficient to clearly define the color and/or information which is intended to be placed on either the drug-containing matrix, the handle, and the label. The drug-containing matrix, the handle, and the label should be suitable for receiving printing, engraving or other information, as such is required by the present invention.

The pharmaceutical dosage unit of the present invention preferably contains a drug-containing matrix. The present invention has particular applicability to a variety of drugs affecting the central nervous system. For example, the present invention may easily be utilized in the administration of opioid agonists (such as fentanyl, alfentanil, sufentanil, lofentanil, and carfentanil), opioid antagonists (such as naloxone and nalbuphine), butyrophenones (such as droperidol and haloperidol); benzodiazepines (such as valium, midazolam, triazolam, oxazolam, and lorazepam); GABA stimulators (such as etomidate); barbiturates (such as thiopental, methohexital, thiamazol, pentobarbital, and hexabarbital); di-isopropylphenols drugs (such as diprivan); and other central nervous system-acting drugs such as levodopa. It will be appreciated that other drugs may also be utilized within the scope of the present invention either singly or in combination. Table 1 lists some of the CNS-acting drugs which may be suitable for incorporation into the dosage form of the present invention, as well as some of the characteristics of those drugs.

TABLE 1

| GENERIC DRUG | DRUG CLASS | DOSE RANGE | |
|---|---|---|---|
| methohexital | barbiturate | 10-500 | mg |
| pentobarbital | barbiturate | 50-200 | mg |
| thiamylal | barbiturate | 10-500 | mg |
| thiopental | barbiturate | 50-500 | mg |
| fentanyl | opioid agonist | 0.05-5 | mg |
| alfentanil | opioid agonist | 0.5-50 | mg |
| sufentanil | opioid agonist | 5-500 | μg |
| lofentanil | opioid agonist | 0.1-100 | μg |
| carfentanil | opioid agonist | 0.2-100 | μg |
| nalbuphine | opioid agonist | 1-50 | mg |
| naloxone | opioid antagonist | 0.05-5 | mg |
| diazepam | benzodiazepine | 1-40 | mg |
| lorazepam | benzodiazepine | 1-4 | mg |
| midazolam | benzodiazepine | 0.5-25 | mg |
| oxazepam | benzodiazepine | 5-40 | mg |
| triazolam | benzodiazepine | 250-1000 | mg |
| droperidol | butyrophenone | 1-20 | mg |
| haloperidol | butyrophenone | 0.5-10 | mg |
| propanidid | substituted eugenol anesth. | 1-10 | mg |
| etomidate | GABA stimulator | 5-60 | mg |
| propofol | substituted phenol | 3-50 | mg |
| ketamine | phencyclidine | 5-300 | mg |

As used herein, the term “pharmaceutically acceptable salt form” broadly refers to pharmaceutically acceptable salts of the drugs described above. As used herein, pharmaceutically acceptable salts includes salts of compounds of the present invention derived from the combination of such compounds with non-toxic acid or base addition salts. Acid addition salts include inorganic acids such as hydrochloric, hydrobromic, hydroiodic, sulfuric, nitric and phosphoric acid, as well as organic acids such as acetic, citric, propionic, tartaric, glutamic, salicylic, oxalic, methanesulfonic, para-toluenesulfonic, succinic, and benzoic acid, and related inorganic and organic acids. Base addition salts include those derived from inorganic bases such as ammonium and alkali and alkaline earth metal hydroxides, carbonates, bicarbonates, and the like, as well as salts derived from basic organic amines such as aliphatic and aromatic amines, aliphatic diamines, hydroxy alkamines, and the like. Such bases useful in preparing the salts of this invention thus include ammonium hydroxide, potassium carbonate, sodium bicarbonate, calcium hydroxide, methylamine, diethylamine, ethylenediamine, cyclohexylamine, ethanolamine and the like.

Of the CNS-acting drugs which may be suitable for incorporation into the drug-containing matrix of the present invention, fentanyl, alfentanil, sufentanil, lofentanil, and carfentanil, or a pharmaceutically acceptable salt form thereof, is preferred. Fentanyl is more preferred.

It should be understood that any suitable pharmaceutically acceptable form of the pharmaceutical agent can be used in the compositions of the present invention. For example, fentanyl, if not used as a free base, can also be used as the fentanyl citrate salt, the fentanyl hydrochloride salt, or any additional pharmaceutically acceptable salt known to one skilled in the art. Fentanyl citrate is preferred.

Fentanyl is preferably included as the citrate salt, in an amount equivalent to from about 50 μg to about 20000 μg of fentanyl free base; preferably from about 50 μg to about 10000 μg of fentanyl free base; preferably from about 50 μg to about 5000 μg of fentanyl free base; preferably from about 50 μg to about 3200 μg of fentanyl free base; more preferably about 100 μg to about 2400 μg of fentanyl free base; more preferably about 200 μg, about 400 μg, about 600 μg, about 800 μg, about 1200 μg, and/or about 1600 μg.

Preferred methods for forming a lozenge-on-a-stick are described in U.S. Pat. No. 4,671,953 to Stanley, et al.; U.S. Pat. No. 4,863,737 to Stanley, et al.; U.S. Pat. No. 5,132,114, to Stanley, et al.; U.S. Pat. No. 5,785,989, to Stanley, et al.; U.S. Pat. No. 5,288,498, to Stanley, et al.; U.S. Pat. No. 5,288,497, to Stanley, et al.; U.S. Pat. No. 5,855,908, to Stanley, et al.; and pending U.S. patent application Ser. No. 10/,771,046, to Hague, et al., filed Feb. 3, 2004; each of which is incorporated herein by reference in their entirety for all purposes.

### EXAMPLE 1

An example of a lozenge-on-a-stick oral transmucosal solid dosage form using the system for identification of the present invention is the ACTIQ® brand of oral transmucosal fentanyl citrate, marketed in the United States under NDA 20-747 filed with the U.S. Food and Drug Administration. In one ACTIQ® pharmaceutical dosage unit, the active ingredient, fentanyl citrate, is intermixed in a sugar-based excipient EMDEX® (spray-crystallized maltose-dextrose spheres) with a buffer (citric acid—di-sodium hydrogen phosphate) flavoring agent and magnesium stearate, and compressed to produce what is essentially a drug-containing matrix, to which a stick including a cylindrical handle has been affixed.

In Example 1 the first indicia printed on the drug-containing matrix includes the brand name “ACTIQ®” as well as the dosage strength “200 mcg”.

In Example 1 the second indicia embossed on the handle includes the generic name “fentanyl” as well as the restriction on use information “Rx”.

In Example 1 the third indicia printed on the label includes the brand name “ACTIQ®”; the generic name “fentanyl”; the dosage strength “200 mcg”; warning information printed as a yellow triangle with an exclamation point printed in the triangle, and color coding correlating to the dosage strength that is printed on the label

### EXAMPLE 2

The lozenge-on-a-stick-type oral transmucosal solid dosage form of Example 1 using the system for identification wherein:
the first indicia includes “ACTIQ®” and the dosage strength “400 mcg”;
the second indicia embossed on the handle includes “fentanyl” and “Rx”; and
the third indicia includes “ACTIQ®”; “fentanyl”; the dosage strength “400 mcg”; a yellow triangle with an exclamation point printed in the triangle, and color coding correlating to the dosage strength that is printed on the label.

### EXAMPLE 3

The lozenge-on-a-stick-type oral transmucosal solid dosage form of Example 1 using the system for identification wherein:
the first indicia includes “ACTIQ®” and the dosage strength “600 mcg”;
the second indicia embossed on the handle includes “fentanyl” and “Rx”; and
the third indicia includes “ACTIQ®”; “fentanyl”; the dosage strength “600 mcg”; a yellow triangle with an exclamation point printed in the triangle, and color coding correlating to the dosage strength that is printed on the label.

### EXAMPLE 4

The lozenge-on-a-stick-type oral transmucosal solid dosage form of Example 1 using the system for identification wherein:
the first indicia includes “ACTIQ®” and the dosage strength “800 mcg”;
the second indicia embossed on the handle includes “fentanyl” and “Rx”; and
the third indicia includes “ACTIQ®”; “fentanyl”; the dosage strength “800 mcg”; a yellow triangle with an exclamation point printed in the triangle, and color coding correlating to the dosage strength that is printed on the label.

### EXAMPLE 5

The lozenge-on-a-stick-type oral transmucosal solid dosage form of Example 1 using the system for identification wherein:
the first indicia includes “ACTIQ®” and the dosage strength “1200 mcg”;
the second indicia embossed on the handle includes “fentanyl” and “Rx”; and
the third indicia includes “ACTIQ®”; “fentanyl”; the dosage strength “1200 mcg”; a yellow triangle with an exclamation point printed in the triangle, and color coding correlating to the dosage strength that is printed on the label.

### EXAMPLE 6

The lozenge-on-a-stick-type oral transmucosal solid dosage form of Example 1 using the system for identification wherein:
the first indicia includes “ACTIQ®” and the dosage strength “1600 mcg”;
the second indicia embossed on the handle includes “fentanyl” and “Rx”; and
the third indicia includes “ACTIQ®”; “fentanyl”; the dosage strength “1600 mcg”; a yellow triangle with an exclamation point printed in the triangle, and color coding correlating to the dosage strength that is printed on the label.

While particular embodiments of the present invention have been illustrated and described herein, it is not intended to limit the invention. Changes and modifications may be made therein within the scope of the following claims.

What is claimed is:

1. A pharmaceutical dosage unit comprising:
a) a stick having opposed first and second ends;
b) a drug-containing matrix being attached to the first end of the stick, wherein the drug-containing matrix displays a first indicia on the surface of the drug-containing matrix;
c) the second end of the stick including a handle, wherein the handle is different in dimensions than the stick; and wherein the handle displays a second indicia; and
d) a label adhered to the surface of the handle, wherein the label displays a third set of indicia.

2. The pharmaceutical dosage unit of claim 1 wherein the drug-containing matrix comprises a drug selected from morphine, codeine, meperidine, methadone, propoxyphene, levorphanol, hydromorphone, oxymorphone, oxycodone, brompton's cocktail, naloxone, naltrexone, pentazocine, butorphanol, nabuphine, buprenorphine, fentanyl, alfentanil, sufentanil, lofentanil, and carfentanil, or pharmaceutically acceptable salt forms thereof.

3. The pharmaceutical dosage unit of claim 1 wherein the drug is selected from fentanyl, alfentanil, sufentanil, lofentanil, and carfentanil, or a pharmaceutically acceptable salt form thereof.

4. The pharmaceutical dosage unit of claim 1 wherein the drug is fentanyl or a pharmaceutically acceptable salt form thereof.

5. The pharmaceutical dosage unit of claim 1 wherein the drug is fentanyl citrate.

6. The pharmaceutical dosage unit of claim 1 wherein the handle has a surface configuration selected from a barrel, a cube, and a rectangular box.

7. The pharmaceutical dosage unit of claim 1 wherein the handle has a surface configuration of a barrel.

8. The pharmaceutical dosage unit of claim 1 wherein the first, second and third indicia convey safety messages.

9. The pharmaceutical dosage unit of claim 1, wherein the first indicia is printed onto the surface of the drug-containing matrix.

10. The pharmaceutical dosage unit of claim 1, wherein the first indicia conveys source of goods information and dosage information.

11. The pharmaceutical dosage unit of claim 1, wherein the third indicia conveys source of goods information and dosage information.

12. The pharmaceutical dosage unit of claim 1, wherein the third indicia conveys information using color coding.

13. The pharmaceutical dosage unit of claim 1, wherein the first and third indicia convey source of goods information.

14. The pharmaceutical dosage unit of claim 1, wherein the first and third indicia convey the same message.

15. The pharmaceutical dosage of claim 1, wherein the second indicia conveys restriction on use information.

16. The pharmaceutical dosage of claim 1, wherein the second indicia is embossed into the handle.

17. The pharmaceutical dosage of claim 1, wherein some or all of the first indicia information is identical to some or all of the second indicia or third indicia; some or all of the second indicia information is identical to some or all of the first indicia or third indicia; and some or all of the third indicia information is identical to some or all of the first indicia or second indicia.

18. The pharmaceutical dosage unit of claim 1 for use in the oral transmucosal delivery of a drug to a patient, in order to induce a sedative, analgesic or anesthetic effect in the patient.

19. A pharmaceutical dosage unit for use in the oral transmucosal or sublingual delivery of a drug to a patient, in order to induce a sedative, analgesic or anesthetic effect in the patient, comprising:
a) a stick having opposed first and second ends;
b) a drug-containing matrix being attached to the first end of the stick, wherein the drug is fentanyl citrate, the drug-containing matrix displays first indicia printed on the surface of the drug-containing matrix, and the first indicia conveys source of goods information and dosage information;
c) the second end of the stick including a handle, wherein the handle is different in dimensions than the stick and has a surface configuration of a cylinder; wherein the handle displays second indicia; the second indicia conveys restriction on use information and the second incicia is embossed into the handle; and
d) a label adhered to the surface of the handle, wherein the label displays third indicia,
wherein the third indicia conveys information to the source of goods and the generic name in addition to dosage information.

20. A pharmaceutical dosage unit for use in the oral transmucosal or sublingual delivery of a drug to a patient, in order to induce a sedative, analgesic or anesthetic effect in the patient, comprising:
a) a stick having opposed first and second ends;
b) a drug-containing matrix being attached to the first end of the stick, wherein the drug is fentanyl citrate, the drug-containing matrix displays first indicia printed on the surface of the drug-containing matrix, and the first indicia conveys the information “ACTIQ” and dosage information selected from “200 mcg”, “400 mcg”, “600 mcg”, “800 mcg”, “1200 mcg”, and “1600 mcg”;
c) the second end of the stick including a handle, wherein the handle is different in dimensions than the stick and has a surface configuration of a cylinder; wherein the handle displays second indicia; the second indicia conveys the information “Rx” and “fentanyl” and the second indicia is embossed into the handle; and
d) a label adhered to the surface of the handle, wherein the label displays third indicia,
wherein the third indicia conveys the information “ACTIQ” and “fentanyl” in addition to dosage information using color coding.
